(19) United States
(12) Patent Application Publication
(10) Pub. No.: US 2021/0174933 A1
(43) Pub. Date: Jun. 10, 2021

(54) Social-Emotional Skills Improvement

(71) Applicant: Social Skills Training Pty Ltd, Indooroopilly (AU)

(72) Inventors: Renae Brooke Beaumont, New York, NY (US); Kathleen Joy Davey, Rochedale South (AU)

(73) Assignee: Social Skills Training Pty Ltd, Indooroopilly (AU)

(21) Appl. No.: 17/113,620

(22) Filed: Dec. 7, 2020

Related U.S. Application Data
(60) Provisional application No. 62/945,351, filed on Dec. 9, 2019.

(51) Int. Cl. G16H 20/70; G16H 50/20; A61B 5/16; G06N 20/00; A63F 13/80

(52) U.S. Cl. CPC G16H 20/70; G16H 50/20; A63F 13/80; A61B 5/165; G06N 20/00; A61B 5/167

(57) ABSTRACT

A method for improving social-emotional skills in a biological subject, including acquiring subject data indicative of a subject response to one or more social cues, deriving metrics from the subject data, including at least one response metric indicative of the subject response, applying the one or more metrics to a computational model to generate a skill state indicator relating to a social-emotional skills state of the subject, the at least one computational model embodying a relationship between social-emotional skill states and the one or more metrics, wherein the at least one computational model is obtained by applying machine learning to reference metrics derived from reference subject data measured for one or more reference subjects and using the skill state indicator to perform a therapeutic intervention to thereby improve the social-emotional skills of the subject.

### CROSS-REFERENCE TO RELATED APPLICATIONS

This application claims the benefit of U.S. Provisional Patent Application No. 62/945,351 filed Dec. 9, 2019 entitled “Social-Emotional Skills Improvement”, which is incorporated by reference herein in its entirety.

### FIELD OF THE INVENTIONS

The present invention relates to a method and apparatus for improving social-emotional skills in a biological subject.

### BACKGROUND OF THE INVENTION

Human relationships are the fabric of our society. Without the ability to connect, empathize and show compassion, we have little hope of building a brighter future for our next generation. The prerequisite skills to create and maintain meaningful relationships are best taught at a young age. Yet, few programs have been shown to effectively teach these skills to children in a manner that translates into improved relationships and lifelong and societal impacts.

Social-emotional learning challenges are estimated to affect 15-20% of children worldwide, contributing to compromised academic performance, mental illness, breakdown of family relationships, an impaired capacity to secure and maintain employment and an increased risk of criminal activity.

“A multi-component social skills intervention for children with Asperger syndrome: the Junior Detective Training Program” by Beaumont R, Sofronoff K, J Child Psychol Psychiatry. 2008 July; 49(7):743-53, describes a study to investigate the effectiveness of a new multi-component social skills intervention for children with Asperger syndrome (AS): The Junior Detective Training Program. This 7-week program included a computer game, in the form of the Secret Agent Society (SAS), which provides a scalable evidence-based solution to engage with children empowering them to build new friendships, and feel happier, calmer and braver, as well as small group sessions, parent training sessions and teacher handouts. SAS was the first gaming-based intervention to successfully empower social-emotional resilience in children with autism. Initial randomized controlled trial findings showed 76% of children with autism with significant social-emotional impairments improved to showing skills within the “normal” range as a result of the program.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavor to which this specification relates.

### SUMMARY OF THE PRESENT INVENTION

In one embodiment, there is a method for improving social-emotional skills in a biological subject, the method including acquiring subject data, the subject data being at least partially indicative of a subject response to one or more social cues, deriving one or more metrics from the subject data, the one or more metrics including at least one response metric at least partially indicative of the subject response, in one or more electronic processing devices, applying the one or more metrics to at least one computational model to generate a skill state indicator relating to a social-emotional skills state of the subject, the at least one computational model embodying a relationship between social-emotional skill states and the one or more metrics, wherein the at least one computational model is obtained by applying machine learning to reference metrics derived from reference subject data measured for one or more reference subjects, and, using the skill state indicator to perform a therapeutic intervention to thereby improve the social-emotional skills of the subject.

In one embodiment, the method further includes generating subject data at least in part based on an assessment of an ability of the subject to recognize the social cues.

In one embodiment, the method further includes exposing the subject to one or more social cues, monitoring subject responses, and, using the subject responses to at least one of: assess the ability of the subject to recognize the social cues, and, train the subject to recognize the social cues.

In one embodiment, the method further includes having the user interact with a computer game implemented using a suitably programmed computer system, the computer game being configured to teach social-emotional skills to the subject, using the computer game to generate game play data, and, using the game play data to derive one or more response metrics.

In one embodiment, the method further includes, in a computer implementing the computer game: presenting the subject with one or more social cues, ascertaining a subject response to the social cues in accordance with user input commands, generating game play data indicative of at least one of: the user input commands, the subject responses, an accuracy of the subject responses, a speed of the subject responses, a degree of participation, demonstrated elements of knowledge, and, selected choices, and, at least one of: progressing the game based on the subject response, and, selectively displaying feedback to the subject based on the subject response.

In one embodiment, the method further includes using one or more sensors to measure subject attributes, and generating one or more metrics using measured subject attributes.

In one embodiment, the method further includes using the one or more sensors to measure subject attributes while the subject is interacting with a computer game implemented using a suitably programmed computer system, the computer game being configured to teach social-emotional skills to the subject playing the computer game.

In one embodiment, the subject data includes at least one of: game play data collected while the subject is interacting with a computer game implemented using a suitably programmed computer system, the computer game being configured to teach social-emotional skills to the subject playing the computer game, details of therapeutic interventions performed on the subject, results of therapeutic interventions performed on the subject, results of one or more assessments of social-emotional skills of the subject; ratings of one or more social-emotional skills of the subject, responses to one or more social cues, subject attributes including one or more of: diagnosed psychological and/or developmental disorders, demographic attributes, physiological attributes, physical attributes, psychological attributes, nutritional information relating to nutrition of the subject, and, medication information relating to medication administered to the subject, recordings of at least one of: conversations, social interactions, and, responses to social cues, and, journal records.

In one embodiment, the method further includes acquiring subject data by at least one of: having the subject interact with a computer game implemented using a suitably programmed computer system, the computer game being configured to teach social-emotional skills to the subject playing the computer game, querying a subject medical history, receiving sensor data from a sensing device, performing an assessment of responses to social cues, using questionnaires or forms completed by the subject, using questionnaires or forms completed by one or more entities delivering therapeutic interventions, and using questionnaires or forms completed by one or more entities assessing social-economic skills of the subject.

In one embodiment, the one or more metrics include at least one of: at least one subject attribute metric indicative of one or more subject attributes of the subject, at least one physiological metric indicative of one or more physiological attributes of the subject, at least one psychological metric indicative of one or more psychological attributes of the subject, at least one physical characteristic metric indicative of one or more physical attributes of the subject, at least one demographic metric indicative of one or more demographic attributes of the subject, a physical activity metric indicative of physical activity of the subject, and, at least one nutritional metric indicative of nutrition ingested by the subject, at least one medication metric indicative of medication administered to the subject, at least one pre-therapy metric, and, at least one post-therapy metric.

In one embodiment, the physiological attributes include at least one of: heart rate, heart rate variability, galvanic skin response, breathing, temperature, a blood pressure, facial expression, gaze, speech, and a presence, absence or degree of one or more disorder states.

In one embodiment, the physical attributes includes at least one of: a subject age, a subject height, a subject weight, a subject sex, and a subject ethnicity.

In one embodiment, the psychological attributes includes at least one of: happy, sad, anxious, angry, tired, and a mental state including at least one of: engaged, focused, disinterested, and bored.

In one embodiment, the physical activity attributes include at least one of: activity type, activity intensity, activity duration, activity location, activity time, and, amount of movement.

In one embodiment, the social-emotional skills state includes at least one of: a diagnosis of a presence, absence or degree of a condition that impacts on social-emotional skills of a subject, a competency level for one or more social-emotional skills, a change in competency level for one or more social-emotional skills, a comparison between a competency level and an expected competency level for one or more social-emotional skills, and, a ranking of social-emotional skills.

In one embodiment, the skill state indicator includes an indication of at least one of: a score for one or more social-emotional skills, the score being indicative of a competency level, results of a comparison between a competency level and an expected competency level for one or more social-emotional skills, a change in competency level for one or more social-emotional skills, a list of one or more social-emotional skills that require improvement, an ordered list indicative of a ranking of social-emotional skills, a recommendation for improving one or more social-emotional skills, one or more tasks designed to improve one or more social-emotional skills, an intervention program for improving one or more social-emotional skills, medication for improving one or more social-emotional skills, and, a diagnosis of a presence, absence or degree of a condition that impacts on social-emotional skills of a subject.

In one embodiment, the therapeutic intervention includes at least one of: controlling a computer game implemented using a suitably programmed computer system, the computer game being configured to teach social-emotional skills to the subject playing the computer game, training the subject, training the subject in accordance with a recommendation, having the subject interact with a computer game implemented using a suitably programmed computer system, the computer game being configured to teach social-emotional skills to the subject playing the computer game, having the subject perform one or more intervention tasks, having the subject complete a treatment program, and, administering medication.

In one embodiment, the method includes generating a report indicative of at least one of: a usage of one or more therapeutic interventions, an adherence to one or more therapeutic interventions, an effectiveness of one or more therapeutic interventions, an effectiveness of one or more entities delivering therapeutic interventions, an effectiveness of one or more entities assessing social-emotional skills, an effectiveness of therapeutic interventions for different classifications of subject, and, a comparison of the effectiveness of different therapeutic interventions.

In one embodiment, the method further includes: assigning the subject to a classification, and, applying the one or more metrics to at least one computational model associated with the classification, the at least one computational model embodying a relationship between social-emotional skills and the one or more metrics for reference subjects assigned to the classification.

In one embodiment, the method further includes assigning the subject to a classification at least in part using subject attributes.

In one embodiment, the method further includes: comparing at least one current metric determined for the subject and at least one previous metric determined for the subject, and, using results of the comparison to track at least one of: change in social-emotional skills of the subject, and, effectiveness of therapeutic interventions.

In one embodiment, the method further includes comparing the at least one current metric and the at least one previous metric by: applying the at least one current metric to the at least one computational model to determine a current indicator, applying the at least one previous metric to the at least one computational model to determine a previous indicator, and, analyzing a difference between the current and previous indicators to determine at least one of: changes in social-emotional skills of the subject, and, an effectiveness of therapeutic interventions.

In one embodiment, the method further includes: for each of a plurality of reference subjects: acquiring reference subject data at least partially indicative of: a reference subject response to one or more social cues, and, an assessment of a reference social-emotional skills state for the reference subject, and, deriving one or more reference metrics from the reference subject data, the one or more reference metrics including at least one response reference metric at least partially indicative of the reference subject response, and, in one or more electronic processing devices, using the reference metrics and the assessed reference social-emotional skills state to train at least one computational model so that the at least one computational model embodies relationships between different social-emotional skill states and the one or more metrics.

In one embodiment, there is a system for improving social-emotional skills in a biological subject, the system including one or more electronic processing devices that are configured to: acquire subject data, the subject data being at least partially indicative of a subject response to one or more social cues, derive one or more metrics from the subject data, the one or more metrics including at least one response metric at least partially indicative of the subject response, apply the one or more metrics to at least one computational model to generate a skill state indicator relating to a social-emotional skills state of the subject, the at least one computational model embodying a relationship between social-emotional skills states and the one or more metrics, wherein the at least one computational model is obtained by applying machine learning to reference metrics derived from reference subject data measured for one or more reference subjects, and, display the skill state indicator to allow the skill state indicator to be used to perform a therapeutic intervention to thereby improve the social-emotional skills of the subject.

In one embodiment, there is a method for use in calculating at least one computational model, the at least one computational model being used for generating a skill state indicator relating to a social-emotional skills state of a biological subject, the method including, in one or more electronic processing devices: for each of a plurality of reference subjects: acquiring reference subject data at least partially indicative of: a reference subject response to one or more social cues, and, an assessment of a reference social-emotional skills state for the reference subject, and, deriving one or more reference metrics from the reference subject data, the one or more reference metrics including at least one response reference metric at least partially indicative of the reference subject response, and, using the reference metrics and the assessed reference social-emotional skills states to train the at least one computational model so that the at least one computational model embodies relationships between different social-emotional skill states and the one or more metrics.

In one embodiment, the one or more processing devices are configured to: select a plurality of reference metrics, train at least one computational model using the plurality of reference metrics, test the at least one computational model to determine a discriminatory performance of the model, and, if the discriminatory performance of the model falls below a threshold, at least one of: selectively retrain the at least one computational model using a different plurality of reference metrics, and, train a different computational model.

In one embodiment, the method further includes: selecting a plurality of combinations of reference metrics, training a plurality of computational models using each of the combinations, testing each computational model to determine a discriminatory performance of the model, and, selecting the at least one computational model with the highest discriminatory performance for use in determining a mental state indicator indicative of a mental state.

In one embodiment, the method further includes: determining one or more reference subject attributes from the reference subject data, and, training the at least one computational model using the one or more reference subject attributes.

In one embodiment, the one or more processing devices are configured to: assign the reference subjects to classifications, and train the at least one computational model using the classifications, so that the at least one computational model embodies a relationship between social-emotional skills and the one or more metrics for reference subjects assigned to a respective classification.

In one embodiment, there is a system for use in calculating at least one computational model, the at least one computational model being used for generating a skill state indicator relating to a social-emotional skills state of a biological subject, the system including one or more electronic processing devices configured to: for each of a plurality of reference subjects: acquire reference subject data at least partially indicative of: a reference subject response to one or more social cues, and, an assessment of a reference social-emotional skills state for the reference subject, and, derive one or more reference metrics from the reference subject data, the one or more reference metrics including at least one response reference metric at least partially indicative of the reference subject response, and, use the reference metrics and the assessed reference social-emotional skills states to train the at least one computational model so that the at least one computational model embodies relationships between different social-emotional skill states and the one or more metrics.

In one embodiment, there is a method for treating social-emotional skills deficits in a biological subject, the method including: acquiring subject data, the subject data being at least partially indicative of a subject response to one or more social cues, deriving one or more metrics from the subject data, the one or more metrics including at least one response metric at least partially indicative of the subject response, in one or more electronic processing devices, applying the one or more metrics to at least one computational model to generate a skill state indicator relating to a social-emotional skills state of the subject, the at least one computational model embodying a relationship between social-emotional skill states and the one or more metrics, wherein the at least one computational model is obtained by applying machine learning to reference metrics derived from reference subject data measured for one or more reference subjects, and, using the skill state indicator to perform a therapeutic intervention to thereby at least partially treat the subject for social-emotional skill deficits.

In one embodiment, there is a system for treating social-emotional skills deficits in a biological subject, the system including one or more electronic processing devices that are configured to: acquire subject data, the subject data being at least partially indicative of a subject response to one or more social cues, derive one or more metrics from the subject data, the one or more metrics including at least one response metric at least partially indicative of the subject response, apply the one or more metrics to at least one computational model to generate a skill state indicator relating to a social-emotional skills state of the subject, the at least one computational model embodying a relationship between social-emotional skills states and the one or more metrics, wherein the at least one computational model is obtained by applying machine learning to reference metrics derived from reference subject data measured for one or more reference subjects, and, display the skill state indicator to allow the skill state indicator to be used to perform a therapeutic intervention to thereby at least partially treat the subject for social-emotional skill deficits.

In one embodiment, there is a method for monitoring social-emotional skills in a biological subject, the method including: acquiring subject data, the subject data being at least partially indicative of a subject response to one or more social cues, deriving one or more metrics from the subject data, the one or more metrics including at least one response metric at least partially indicative of the subject response, in one or more electronic processing devices, applying the one or more metrics to at least one computational model to generate a skill state indicator relating to a social-emotional skills state of the subject, the at least one computational model embodying a relationship between social-emotional skill states and the one or more metrics, wherein the at least one computational model is obtained by applying machine learning to reference metrics derived from reference subject data measured for one or more reference subjects, and, using the skill state indicator to monitor the social-emotional skills of the subject.

In one embodiment, there is a system for monitoring social-emotional skills in a biological subject, the system including one or more electronic processing devices that are configured to: acquire subject data, the subject data being at least partially indicative of a subject response to one or more social cues, derive one or more metrics from the subject data, the one or more metrics including at least one response metric at least partially indicative of the subject response, apply the one or more metrics to at least one computational model to generate a skill state indicator relating to a social-emotional skills state of the subject, the at least one computational model embodying a relationship between social-emotional skills states and the one or more metrics, wherein the at least one computational model is obtained by applying machine learning to reference metrics derived from reference subject data measured for one or more reference subjects, and, display the skill state indicator to allow the skill state indicator to be used to monitor social-emotional skills in the subject.

In one embodiment, there is a method for monitoring social-emotional skills therapies, the method including: for each of a plurality of subjects, acquiring subject data, the subject data being at least partially indicative of: a subject response to one or more social cues, and, an indication of one or more therapeutic interventions, deriving one or more metrics from the subject data, the one or more metrics including at least one response metric at least partially indicative of the subject response, and, using the metrics to monitor social-emotional skills in the subject, and, analyzing the metrics to assess social-emotional skills therapies.

In one embodiment, the method further includes using machine learning to analyze the metrics.

In one embodiment, the method further includes: using the metrics to identify changes in social-emotional skills, and, using the changes in social-emotional skills to assess an effectiveness of therapies.

In one embodiment, the method further includes generating a report indicative of at least one of: a usage of one or more therapeutic interventions, an adherence to one or more therapeutic interventions, an effectiveness of one or more therapeutic interventions, an effectiveness of one or more entities delivering therapeutic interventions, an effectiveness of one or more entities assessing social-emotional skills, an effectiveness of therapeutic interventions for different classifications of subject, and, a comparison of the effectiveness of different therapeutic interventions.

In one embodiment, there is a system for monitoring social-emotional skills therapies, the system including one or more processing devices configured to: for each of a plurality of subjects, acquire subject data, the subject data being at least partially indicative of: a subject response to one or more social cues, and, an indication of one or more therapeutic interventions, derive one or more metrics from the subject data, the one or more metrics including at least one response metric at least partially indicative of the subject response, and, using the metrics to monitor social-emotional skills in the subject, and, analyze the metrics to assess social-emotional skills therapies.

It will be appreciated that the embodiments of the invention and their respective features can be used in conjunction and/or independently, and reference to separate embodiments is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:

FIG. 1A is a flow chart of an example of a method for improving social-emotional skills in a biological subject;

FIG. 1B is a flow chart of an example of a method for training a computational model;

FIG. 2 is a schematic diagram of an example of a network architecture;

FIG. 3 is a schematic diagram of an example of a processing system;

FIG. 4 is a schematic diagram of an example of a client device; and,

FIGS. 5A to 5C are a flow chart of a further example of a process for improving social-emotional skills in a biological subject.

### DETAILED DESCRIPTION OF THE PREFERRED EMBODIMENTS

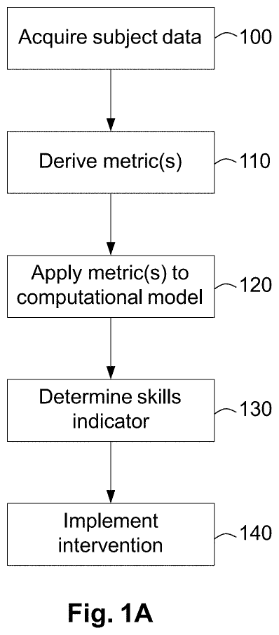

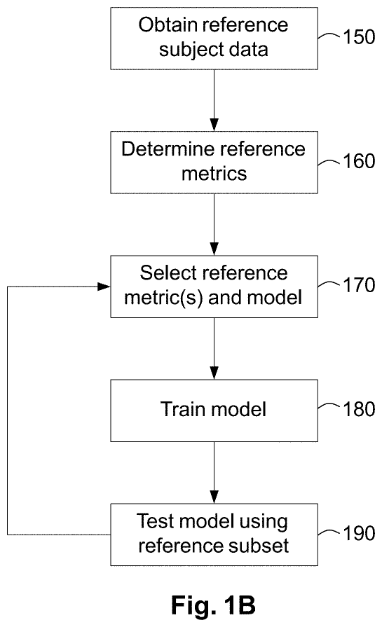

An example of a method for improving social-emotional skills in a biological subject will now be described with reference to FIG. 1A.

For the purpose of illustration, it is assumed that the process is performed at least in part using one or more electronic processing devices forming part of one or more processing systems, such as computer systems, servers, or the like, which are optionally connected to one or more client devices, such as mobile phones, portable computers, tablets, or the like, via a network architecture, as will be described in more detail below. Furthermore, for ease of illustration the remaining description will refer to a processing device, but it will be appreciated that multiple processing devices could be used, with processing distributed between the devices as needed, and that reference to the singular encompasses the plural arrangement and vice versa.

For the remaining description the term “social-emotional skills” is used to describe the knowledge, attitudes, and skills necessary for individuals to recognize and control their emotions and behaviors, particularly in the context of interaction with other individuals, including in establishing and maintaining positive relationships with other individuals. A specific definition of social-emotional skills is provided in the Devereux Students Strengths Assessment (DESSA) comprising eight social and emotional competencies, including:
Self-Awareness—A child's realistic understanding of her/his strengths and limitations and consistent desire for improvement.
Self-Management—A child's success in controlling his or her emotions and behaviors, to complete a task or succeed in a new or challenging situation.
Social-Awareness—A child's capacity to interact with others in a way that shows respect for their ideas & behaviors, recognizes her/his impact on them, and uses cooperation and tolerance in social situations.
Relationship Skills—A child's consistent performance of socially acceptable actions that promote and maintain positive connections with others.
Goal-Directed Behavior—A child's initiation of, and persistence in completing tasks of varying difficulty.
Personal Responsibility—A child's tendency to be careful and reliable in her/his actions and in contributing to group efforts.
Decision-Making—A child's approach to problem solving that involves learning from others and her/his own previous experiences, using her/his values to guide her/his action, and accepting responsibility for her/his decisions.
Optimistic Thinking—A child's attitude of confidence, hopefulness, and positive thinking regarding herself/himself and her/his life situations in the past, present, and future.

The term “social cue” is used to refer to a verbal or non-verbal hint, including body language, eye movement, facial expression, vocal intonations, or the like, which can be positive or negative, and which typically guide conversation and other social interactions.

The term “reference subject” is used to refer to one or more individuals in a sample population, with “reference subject data” being used to refer to data collected from the reference subjects. The term “subject” refers to any individual that is being assessed for the purpose of monitoring and/or improving the subject's social-emotional state, with “subject data” being used to refer to data collected from the subject. The reference subjects and subjects are typically humans.

It will be appreciated that the above described terms should not be interpreted narrowly, and any reasonable interpretation should be applied to these terms.

In this example, at step 100 subject data is obtained which is at least partially indicative of a subject response to one or more social cues, and which may also include a range of other different types of data, such as details of subject attributes, assessments of social-emotional skills, details of interventions performed on the subject, demographic information, or the like, and additional examples are described in more detail below.

The subject data could be obtained in any appropriate manner depending on the nature of the subject data. Typically this involves exposing the subject to social cues and then monitoring a response of the subject, either manually and/or using sensing devices, and using this to generate the subject data. Thus, for example, data could be received from a monitoring device and/or one or more sensors, which monitor responses of the subject, or could be received via a user interface, in accordance with user input commands. Additionally and/or alternatively, the subject data could be retrieved from a data store such as a database, supplied by an individual other entity assessing the subject, provided by a computer system in response to the subject interacting with a computer game, or the like, and additional examples are described in more detail below.

At step 110 the subject data is analyzed to determine at least one metric. The metric(s) used will vary depending upon a range of factors, such as the subject data captured, the computational model(s) used to perform analysis, or the like. Typically however, the metric(s) include at least one response metric at least partially indicative of the subject response, such as whether the subject accurately identified a social cue, how long the identification took, and how the subject responded to the social cue.

At step 120 the one or more metrics are applied to at least one computational model. For the remainder of the description, the term computational model will be understood to encompass one or more models and it will be understood that reference to a singular model is not intended to be limiting and could encompass using multiple models.

The computational model typically embodies a relationship between social-emotional skill states and the one or more metrics. In this regard, the social-emotional skill state is intended to represent the ability of the subject to use social-emotional skills in order to interact with other individuals, and as such could represent a presence, absence or degree of a condition that impacts on social-emotional skills of a subject, a competency level for one or more social-emotional skills, or the like.

The computational model can be obtained by applying machine learning to reference metrics derived from one or more reference subjects having known social-emotional skill states and an example of this process will be described below with reference to FIG. 1B.

The computational model is used to determine a skill state indicator related to the social-emotional skill state at step 130. The skill state indicator could be indicative of a wide range of information, and could, for example, include information regarding competency levels of different social-emotional skills, diagnosis of a disease or other condition, recommendations for interventions, such as training or other tasks to mitigate skills deficiencies, or the like. Thus, for example, the skill state indicator could indicate particular social-emotional skills that could be improved in the subject, and provide recommendations regarding one or more tasks that could assist the subject in improving those skills.

At step 140, the skill state indicator can be used in order to perform a therapeutic intervention to thereby improve the social-emotional skills of the subject. For example, a trainer, teacher or other individual could identify deficient social-emotional skills, and then perform training in order to specifically target those skills deficiencies and thereby improve the overall social-emotional skills of the subject, with this optionally being performed based on recommendations in the skill state indicator.

Accordingly, it will be appreciated that the above described method utilizes a computational model in order to assess social-emotional skills of a subject. This allows a more objective assessment of a subject's capabilities, and hence deficiencies, to be used in order to guide therapeutic interventions, thereby improving outcomes. Furthermore, the computational model can be used to recommend interventions that can successfully target specific skills deficiencies, and optionally tailor these based on additional information, such as demographic information or similar, allowing interventions to be targeted to specific subjects, thereby further improving outcomes.

A number of further features will now be described.

In one example, the method includes generating the subject data at least in part based on an assessment of an ability of the subject to recognize the social cues. The assessment is typically performed by exposing the subject to one or more social cues, with subject responses being monitored to allow the subject responses to be used to assess the ability of the subject to recognize the social cues. This can additionally, and/or alternatively, be used to help train the subject to recognize social cues, for example by providing feedback to the subject regarding whether their identification of the social cue was correct.

This process can be performed in a learning environment, such as a classroom, where the subject interacts with other people, for example as part of role playing scenarios, general day to day interactions, or the like. In this example, the assessment can be performed by an overseeing individual, such as a teacher or similar, who may collect information regarding the interactions for example, using paper based forms, or a user interface on a suitably programmed computer system, client device, or similar. Additionally and/or alternatively monitoring can be performed using equipment, such as cameras and/or physiological sensors.

In another example, this process is performed at least in part by having the user interact with a computer game implemented using a suitably programmed computer system. The computer game is typically configured to teach social-emotional skills to the subject, and may achieve this by presenting the subject with one or more social cues and ascertaining subject responses to the social cues in accordance with user input commands. Thus, for example, the computer game can present role playing scenarios to the subject, asking the subject to provide input in response to the role playing scenario. The response can be used to interact with, and hence progress the game and can also allow the computer game to selectively display feedback to the subject based on the subject response, for example to indicate whether the subject accurately identified a social cue, thereby assisting in training the subject.

In general, the game aims to make subjects feel happier, calmer and braver and to make and keep friends, and teaches skills such as emotion recognition in others from face, voice and body clues, emotion recognition in oneself from body clues and thoughts, relaxation ‘gadgets’, friendship values and strengths, steps for talking, playing and working with others, how to cope with mistakes and feelings of confusion, how to detect the difference between friendly joking and mean teasing, and how to prevent and manage bullying.

As part of this process, the computer game generates game play data, which can be indicative of aspects of the interaction, such as the user input commands, the subject responses, an accuracy of the subject responses, a speed of the subject responses, a degree of participation, and demonstrated elements of knowledge and/or selected choices. The game play data can be used to form part of the subject data, and hence be used to derive one or more response metrics.

It will be appreciated that in one example, the computer game can include and/or be based on, the Secret Agent Society (SAS) computer game described above. Using a computer game in this manner provides a number of advantages. For example, the response reward mechanisms associated with computer games can encourage use, thereby allowing a greater amount of data to be collected. Additionally, as data is collected during the game play process automatically, a wide range of data can be collected that may not otherwise be collectable, such as the time taken for the subject to respond to a cue. Furthermore, the data is highly objective, and avoids the use of subject assessment, which can in turn lead to inconsistencies the data.

In another example, the method includes using one or more sensors to measure attributes of the subject and generate the one or more metrics using measured subject attributes. The subject attributes can include physiological and/or psychological attributes, and could include information such as heart rate, breathing rate, or the like, and additional examples are described below. The collection of such data can be performed at any time, but in one example is performed while the subject is interacting with the computer game as described above, thereby providing an additional layer of objective data that can accompany the collected game play data.

Where sensors are used, these are typically incorporated into a monitoring system. The nature of such a monitoring system will vary depending upon the preferred implementation. In one example, the monitoring system includes a monitoring device having at least one sensor and a monitoring device processor that generates sensor data in accordance with signals from the sensor. The sensor data is typically indicative of physiological attributes, such as a heart rate, physical activity of the subject, or the like, depending on the nature of the sensor employed. Additionally, speech analysis could be performed, using a microphone to capture speech, with this being analyzed to detect variations in pitch, tone or the like.

In one example, the monitoring device is in the form of a wearable monitoring device which could include a wrist mounted heart rate monitor, including a suitable heart rate detection mechanism, such as an optical based system for detection of wrist pulse. Physical activity can be determined through the use of accelerometers or gyroscopes and may be incorporated into a wearable device. In one particular example, the monitoring device includes a wrist mounted smart watch or similar, with an optional chest strap for improved heart rate sensing. Additionally and/or alternatively electrode based detection can be used to acquire Galvanic Skin Response signals, or the like.

It will be appreciated that the form factor of the monitoring device and the particular sensing provided can vary depending on the circumstances in which the monitoring device is to be used. For example, when used in a home environment, the monitoring device is typically a wearable device, with more limited sensing capabilities, often limited to optical and/or movement sensing, whereas if the device is adapted to be used in a clinical environment, electrode based systems can be used for capturing ECG and EEG signals, for greater accuracy.

In one example, the monitoring device is adapted to upload sensor data directly to the one or more processing devices, which could be situated remotely in a cloud based environment, or locally, for example on a computer system. In another example, the monitoring system includes a client device, such as a smartphone, tablet or computer system, that receives sensor data from the monitoring device and uses the sensor data to generate captured subject data. The captured subject data typically includes a subject identifier indicative of an identity of the subject, which could be a device identifier of either the client device or monitoring device, which is associated with the subject, or could be a user name or real name of the subject, or a unique identifier associated with the subject, as well as any relevant physiological data, such as heart rate data indicative of the measured heart rate, or the like. The client device can also be adapted to perform at least preliminary processing of the sensor data, such as filtering of signals, derivation of parameters from the signals, or the like.

The captured subject data is transferred to the one or more processing devices, allowing these to incorporate the captured subject data into subject data using the identifier. Thus, the processing device can identify the stored subject data associated with the respective subject using the identifier, before updating the stored subject data with the captured subject data.

Thus, in the above examples, the client device acts to acquire sensor data from the monitoring device, perform optional processing, and add an identifier, transferring this as captured subject data to the one or more processing devices, which are typically in the form of remote servers, allowing the subject data to be consolidated and processed remotely. In this example, the client device, which is typically a smart phone or tablet of the subject, effectively acts to forward the captured data to the processing devices for analysis as required.

The above described arrangement provides a number of benefits. For example, this ensures subject data is stored centrally, allowing this to be used in training computational models. This in effect allows data from multiple subjects to be mined so that more accurate models can be constructed thereby improving the discriminatory power of the system. Additionally, the client device can be used to leverage existing hardware functionality in order to reduce the hardware requirements of the monitoring device.

In a further example, the client device can be used to collect additional information, such as subject attributes. In this example, the client device can display one or more questions, generating the captured subject data at least in part in response to user input commands provided in response to the one or more questions. This allows the user to be presented with questions, which can in turn assist in assessment of the subject's social-emotional skill state, including capturing information relating to symptoms, as well as other information, such as questions regarding subject attributes, dietary habits, medication consumed, or the like.

Subject data can also be collected from other sources, such as by querying a subject medical history, by receiving sensor data from a sensing device, by performing an assessment of responses to social cues, by using questionnaires or forms completed by the subject, by using questionnaires or forms completed by one or more entities delivering therapeutic interventions or by using questionnaires or forms completed by one or more entities assessing social-emotional skills of the subject.

In one example, the subject could supply information the first time monitoring is performed, for example when registering to undergo a monitoring process, and/or periodically, for example each time monitoring is performed, at set time intervals, such as once a week or month, or the like. It will be appreciated that regular updates of subject attributes are typically used for more variable attributes, such as details of medical symptoms or the like, whilst attributes that are more static may be determined on a one-off basis. It will also be appreciated that the processing devices could determine at least some of the subject attributes based on a subject medical history, for example by retrieving or querying a patient medical record.

Where subject data is subjective, for example when collected based on observation by individuals, data will typically be labeled, for example classifying emotion (11 options) and degree of intensity (scale 0.1 to 1). Accurate emotional state information is typically required at a resolution of 1-5 seconds and so transcripts of participant speech can be used to validate subject assessments.

From the above, it will be appreciated that the subject data can include a wide range of different information, including but not limited to game play data, details of therapeutic interventions performed on the subject, results of therapeutic interventions performed on the subject, results of one or more assessments of social-emotional skills of the subject, ratings of one or more social-emotional skills of the subject or responses to one or more social cues. The subject data can include subject attributes such as diagnosed psychological and/or developmental disorders, demographic attributes, physiological attributes, physical attributes, psychological attributes, nutritional information relating to nutrition of the subject or medication information relating to medication administered to the subject. The subject data may also include recordings of one or more conversations, social interactions, or responses to social cues, journal records, or the like.

It will be appreciated from this that a wide range of metrics can be derived from the subject data and examples of such metrics include, but are not limited to subject attribute metric(s) indicative of one or more subject attributes of the subject, physiological metric(s) indicative of one or more physiological attributes of the subject, psychological metric(s) indicative of one or more psychological attributes of the subject, physical characteristic metric(s) indicative of one or more physical attributes of the subject, demographic metric(s) indicative of one or more demographic attributes of the subject, physical activity metric(s) indicative of physical activity of the subject, nutritional metric(s) indicative of nutrition ingested by the subject, medication metric(s) indicative of medication administered to the subject, or the like. Metrics can include pre-therapy metric(s) collected prior to the subject undergoing therapy and/or post-therapy metric(s) collected after a therapeutic intervention has been performed.

The physiological attributes and associated metrics can include any one or more of heart rate, heart rate variability, galvanic skin response, breathing, temperature, a blood pressure, gaze, speech, a presence, absence or degree of one or more disease states, or the like. The physical attributes and associated metrics typically include one or more of a subject age, a subject height, a subject weight, a subject sex or a subject ethnicity, whilst the psychological attributes and associated metrics can include an indication of whether the subject is one or more of happy, sad, anxious, angry, tired, or an indication of a social-emotional skill state such as engaged, focused, disinterested, bored, or the like. The physical activity attributes and metrics can include an activity type, activity location, activity duration, activity intensity, activity time and amount of movement.

The analysis is also typically performed to take into account subject attributes, such as subject characteristics, demographic information, or the like. In this example, the one or more processing devices can use the one or more subject attributes to apply the computational model so that the at least one metric is assessed based on reference metrics derived for one or more reference subjects having similar attributes to the subject attributes. This can be achieved in a variety of ways, depending on the preferred implementation, and can include selecting metrics and/or one of a number of different computational models at least in part depending on the subject attributes. For example, this could include assigning the subject to a classification based on physical and/or demographic attributes, and then applying the one or more metrics to at least one computational model associated with the classification, so that the computational model embodies a relationship between social-emotional skills and the one or more metrics for reference subjects also assigned to the classification. As a result, this allows the computational model to be specific to subjects within the classification. Irrespective of how this is achieved, it will be appreciated that taking into account subject attributes can further improve the discriminatory performance and/or recommendations regarding interventions, by taking into account that subjects with different attributes may have different inherent social-emotional skills, may respond differently to same therapies, or the like.

The social-emotional skills state can include any information that is useful in understanding the social-emotional ability of the subject. In one example, the skill state includes a diagnosis of a presence, absence or degree of a condition that impacts on social-emotional skills of a subject, a competency level for one or more social-emotional skills, a change in competency level for one or more social-emotional skills, a comparison between a competency level and an expected competency level for one or more social-emotional skills or a ranking of social-emotional skills. It will be appreciated that other measures could also be used and the above examples are not intended to be limiting.

In one example, the skill state indicator includes an indication of one or more of a score for one or more social-emotional skills, the score being indicative of a competency level, results of a comparison between a competency level and an expected competency level for one or more social-emotional skills, a change in competency level for one or more social-emotional skills, a list of one or more social-emotional skills that require improvement, an ordered list indicative of a ranking of social-emotional skills, a recommendation for improving one or more social-emotional skills, one or more tasks designed to improve one or more social-emotional skills, an intervention program for improving one or more social-emotional skills, medication for improving one or more social-emotional skills or a diagnosis of a presence, absence or degree of a condition that impacts on social-emotional skills of a subject. Thus, it will be appreciated that the skill state indicator can indicate a current level of social-emotional skills, as well as recommendations, and in particular recommendations or therapeutic interventions, which can be used in order to improve the social-emotional skills of the subject.

In one example, the one or more processing devices display a representation of the skill state indicator, such as an alphanumerical and/or graphical representation of the indicator. The processing device can also store the skill state indicator for subsequent retrieval or provide the skill state indicator to a client device for display. Thus, it will be appreciated that the skill state indicator can be used in a variety of manners, allowing this to be used to perform therapeutic interventions.

The nature of the therapeutic intervention performed will vary depending on the preferred implementation and could include one or more of training or teaching the subject, optionally in accordance with a recommendation. The therapeutic intervention could involve having the subject interact with the above described computer game, and optionally controlling the game to thereby more effectively target the needs of the subject, for example tailoring the scenarios presented to the subject to thereby more effectively address issues. For example, if the subject has a particular social-emotional deficiency, the game can be adapted to focus on that particular deficiency, with the manner in which this is performed by adapted based on attributes of the subject, so that this trains the subject in the most effective manner possible. Alternatively, the therapeutic intervention could involve having the subject perform one or more intervention tasks, having the subject complete a treatment program and/or administering medication to the subject.

The system can also be utilized to perform longitudinal monitoring in which changes in a subject's social-emotional skill state are tracked. This can be performed for the purpose of monitoring of progression of therapeutic interventions. In this example, a comparison is performed between at least one current metric determined for the subject and at least one previous metric determined for the subject, with results of the comparison being used to track a change in a social-emotional skill state. The comparison can be achieved by directly comparing particular metrics, but it will be appreciated that this tends to provide little in the way of guidance regarding the progression of the social-emotional skill state. Accordingly it is more typical for the current metric to be applied to the computational model to determine a current skill state indicator indicative of a current social-emotional skill state, and to apply the previous metric to the computational model to determine a previous skill state indicator indicative of a previous social-emotional skill state, and then analyze a difference between the current and previous skill state indicators to determine the change in social-emotional skill state.

It will be appreciated that in practice, the system will maintain a record of social-emotional skill state indicators, so that a sequence of skill state indicators can be used to demonstrate changes in the social-emotional skill state over time. In one example, such trending can be plotted, allowing an allied health practitioner or educator to readily observe changes in social-emotional skill state. For example, this may demonstrate improvements in social-emotional skill states as therapies progress.

In addition to generating a skill state indicator associated with the particular subject, the system can be used to produce a wider range of reports. In this regard, the system is typically used to monitor and generate skill state indicators for a wide range of different subjects, often with multiple subjects associated with multiple different facilities, such as schools, clinics, or the like. Accordingly, in one example the system can be used to perform data mining across all of this information, and generate reports such as details of usage of one or more therapeutic interventions, adherence to one or more therapeutic interventions, an effectiveness of one or more therapeutic interventions, an effectiveness of one or more entities, such as individuals, or facilities, at delivering therapeutic interventions, an effectiveness of one or more entities assessing social-emotional skills, an effectiveness of therapeutic interventions for different classifications of subject, a comparison of the effectiveness of different therapeutic interventions, or the like.

These reports can therefore be used to identify therapies, entities or individuals that are more or less effective, allowing this information to be used to improve outcomes. For example, if a facility is minimally effective at improving the outcomes of subjects within that facility, then this can be used to review the facility and identify where improvements can be made. Similarly, less effective therapies could be replaced by more effective therapies, thereby improving outcomes for all subjects.

The above described approaches use a computational model in order to generate the skill state indicator, and an example of a process for generating such a model will now be described with reference to FIG. 1B.

In this example, reference subject data is obtained at step 150, which is at least partially indicative of a subject response to one or more social cues, and which may also include a range of other different types of data, such as details of subject attributes, assessments of social-emotional skills, details of interventions performed on the subject, demographic information, or the like.

At step 160 the reference subject data is analyzed to determine at least one reference metric including at least one response reference metric at least partially indicative of the reference subject response.

Steps 150 and 160 are largely analogous to steps 100 and 110 described with respect to obtaining and analyzing subject data of a subject, and it will therefore be appreciated that these can be performed in a largely similar manner, and hence will not be described in further detail.

In contrast however, as the reference subject data is used in training a computational model, the reference subject data will also include an assessment of a reference social-emotional skills state for the reference subject, which may have been performed by a qualified professional or other expert assessor. This allows a relationship between metrics and a social emotional skill state to be derived. The reference subject data may also include other information, such as details of interventions performed on the reference subject, together with a measure of the relative success of the interventions, which can in turn be used to guide the generation of recommendations.

Additionally, when using the reference subject data to train the computational model, it will be typical to determine reference metrics for all available metrics, rather than just selected ones of the metrics, allowing this to be used in order to ascertain which of the metrics are most useful in discriminating between different social-emotional skill states.

It will also be appreciated that the reference subject data can be derived from subject data collected for subjects when an assessment is being performed as described with reference to FIG. 1A. In this instance, the social-emotional skill state determined by applying the above described method can be used as part of the reference data, as long as the assessment has subsequently been verified.

At step 170 a combination of the reference metrics and a generic computational model are selected, with the reference metrics and identified social-emotional skill state for a plurality of reference subjects being used to train the model at step 180. The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, K-means clustering, Naïve Bayes Classifier Algorithms, Nearest Neighbor learning, or the like. As such schemes are known, these will not be described in any further detail.

Accordingly, the above described process provides a mechanism to develop a computational model that can be used in generating a social-emotional skill state indicator using the process described above with respect to FIG. 1A.

In addition to simply generating the model, the process typically includes testing the model at step 190 to assess the discriminatory performance of the trained model. Such testing is typically performed using a subset of the reference subject data, and in particular, different reference subject data to that used to train the model, to avoid model bias. The testing is used to ensure the computational model provides sufficient discriminatory performance. In this regard, the discriminatory performance is typically based on an accuracy, sensitivity, specificity and AUROC, with a discriminatory performance of at least 70% typically being required in order for the model to be used.

It will be appreciated that if the model meets the discriminatory performance, it can then be used in determining a social-emotional skill state indicator using the process outlined above with respect to FIG. 1A. Otherwise, the process returns to step 170 allowing different metrics and/or models to be selected, with training and testing then being repeated as required.

Accordingly, the above described approach provides a mechanism to derive the computational model that can be used in assessing the social-emotional skill state of a subject. In this regard, the process involves collecting reference subject data, equivalent to subject data, for a plurality of reference subjects for which a variety of different social-emotional skill states have been identified. The collected reference subject data is used to calculate reference metrics, which are then used to train the computational model so that the computational model can discriminate between different social-emotional skill states.

A number of further features will now be described.

In one example, the process typically involves having the one or more processing devices select a plurality of reference metrics, typically selected as a subset of available metrics, train a computational model using the plurality of reference metrics, test the computational model to determine a discriminatory performance of the model and if the discriminatory performance of the model falls below a threshold then selectively retrain the computational model using a different plurality of reference metrics and/or train a different computational model. Accordingly, it will be appreciated that the above described process can be performed iteratively utilizing different metrics and/or different computational models until a required degree of discriminatory power is obtained.

As an alternative, the one or more processing devices can select a plurality of combinations of reference metrics, train a plurality of computational models using each of the combinations, test each computational model to determine a discriminatory performance of the model and select the computational model with the highest discriminatory performance for use in determining a social-emotional skill state indicator indicative of a social-emotional skill state.

In addition to using the metrics to train the models, the training can also be performed taking into account reference subject attributes, so that models are specific to respective reference subject attributes or can take the subject attributes into account when determining the social-emotional skill state. In one example, this process involves having the one or more processing devices perform clustering using the reference subject attributes to determine clusters of reference subjects having similar reference subject attributes, for example using a clustering technique such as k-means clustering, and then training the computational model at least in part using the reference subject clusters. For example clusters of reference individuals suffering from a particular anxiety diagnosis could be identified, with this being used to train a computational model to assess social-emotional skill states for subjects also having the respective particular anxiety diagnosis. It will be appreciated however that any suitable technique could be used.

In one example, the above described approach can be implemented using standard benchmarking metrics used in the research community, including both quantitative and qualitative metrics. The system-level metrics of focus include, how often does the system:
Accurately detect moments of distress?
Prompt the use of relevant self-soothing strategies?
Accurately assess the quality of a conversation?
Give relevant social cues feedback?
Accurately assess facial emotion?
Accurately track and assess the user's social and emotional skill application?

In one example, validation is performed to determine accuracy through trained human assessment and feedback, including:
User feedback: answering post-action questions (“Was this right? Did this work?”)
SAS facilitator and clinical assessment: reviewing automated assessments both at the whole-of-functioning level and spot-checking specific instances
Supporter feedback: certain non-sensitive items can be validated by sharing with a wider audience, for instance facial emotion classifications of third-party media

Accordingly, the above described techniques provide a mechanism for training one or more computational models to discriminate between different social-emotional skill states using a variety of different metrics, and then using the model(s) to generates social-emotional skill state indicators indicative of the likelihood of a subject having a particular social-emotional skill state, thereby assisting in the assessment of social-emotional skill states.

In another example, the system can be used for diagnosing and/or treating social-emotional conditions, deficits or impairments, including but not limited to Autism, developmental or psychological conditions, ADHD, Anxiety Disorders, Pervasive Developmental Disorder-Not Otherwise Specified (PDD-NOS), social pragmatic communication disorder, tic disorder, peer relationship difficulties or social anxiety. The system can also be used solely for the purpose of monitoring, for example without any treatment or intervention component. This could be used in monitoring progression of social-emotional skill development within a subject, assessing the effectiveness of interventions, or the like.

The system could further be used to identify factors contributing to social-emotional skills deficiencies, which can arise for a number of reasons.

For example, deficits in skilled social behavior can be the result of a failure to acquire social skills, a failure to produce social skills, or a combination of these factors. Key reasons children may fail to produce socially skilled behaviors include difficulties with social cognition (misinterpreting social situations or social problem solving), difficulties with emotion regulation (not being in an emotional state to produce the appropriate social skills, or access social cognition skills) and insufficient opportunities to interact with peers (e.g., peer rejection, neglect or victimization)

Other common contributors to social skill deficits in children include difficulties recognizing how others feel from their facial, postural, voice tone and contextual cues, and aspects of social cognition such as attention, information processing, decision-making and planning abilities. These challenges are particularly prevalent amongst children who struggle with anxiety and anger management. For example, children who are anxious in social situations are prone to social perception and interpretation difficulties due to self-focused attention, gaze aversion, concentration problems and difficulties in recognizing emotions in others, and there is some evidence to suggest they are biased towards interpreting ambiguous information negatively

Children who display aggressive behaviors often have notable gaps in their emotional knowledge, problems in attending to and encoding social information, hostile attributional biases and challenges with social problem-solving. Children prone to anxiety and depression are more likely to have difficulty regulating their emotions, and be shy and socially withdrawn relative to their peers. Children experiencing anxiety are more vulnerable to peer relationship problems than their non-anxious counterparts. This can occur through showing greater distress (e.g., crying) when upset or victimized, or through engaging in potentially annoying behaviors, such as excessive reassurance-seeking, compulsive behaviors, or dinginess.

Thus, by collecting historical data regarding subjects, the system can be configured to analyze the data using machine learning, to identify patterns that can lead to specific social-emotional deficits, which in turn can assist in treating such deficits more effectively.

In another example, the above described system and method can be used for monitoring social-emotional skills therapies, for example to assess the effectiveness of therapies and or entities administering therapies.

In this example, the approach involves acquiring subject data for each of a plurality of subjects, with the subject data including a subject response to one or more social cues and an indication of one or more therapeutic interventions. Metrics are then derived from the subject data, with these being used to monitor social-emotional skills in the subject. Whilst this can be performed using any suitable approach, this is typically performed using the above described techniques, which will not therefore be described in any further detail.

Additionally, as the system is able to capture data from multiple subjects, typically undergoing multiple different therapies, which are in turn administered by multiple entities, this allows the metrics to be used in assessing the effectiveness of therapies and or entities administering therapies.

Accordingly, this provides a mechanism to allow data collected from multiple subjects (and/or reference subjects) in order to assess the effectiveness of therapies and/or individuals or other entities administering therapies.

In one example, this is performed at least in part using machine learning to analyze the metrics and/or changes in social-emotional skill states. In one particular example, this is achieved using the metrics to identify changes in social-emotional skills using the techniques described above, and then using the changes in social-emotional skills to assess effectiveness of therapies. Thus, this process could be achieved by having the processing device perform clustering using subject attributes and therapy details to determine clusters of subjects having similar subject attributes, which have undergone similar therapies. Changes in social-emotional skills in response to therapies can then be monitored for different groups, with this being used to assess the effectiveness of the therapies.

In one example, this is used to generate reports, which can provide information such as details of usage of one or more therapeutic interventions, adherence to one or more therapeutic interventions, an effectiveness of one or more therapeutic interventions, an effectiveness of one or more entities, such as individuals, or facilities, at delivering therapeutic interventions, an effectiveness of one or more entities assessing social-emotional skills, an effectiveness of therapeutic interventions for different classifications of subjects, a comparison of the effectiveness of different therapeutic interventions, or the like.

Social-emotional skills deficits and their treatment using a game are described in more detail in “A Novel Intervention for Child Peer Relationship Difficulties: The Secret Agent Society” by Renae B. Beaumont, Roxana Pearson, Kate Sofronoff, Journal of Child and Family Studies 26 Jun. 2019, the content of which is incorporated herein by reference.

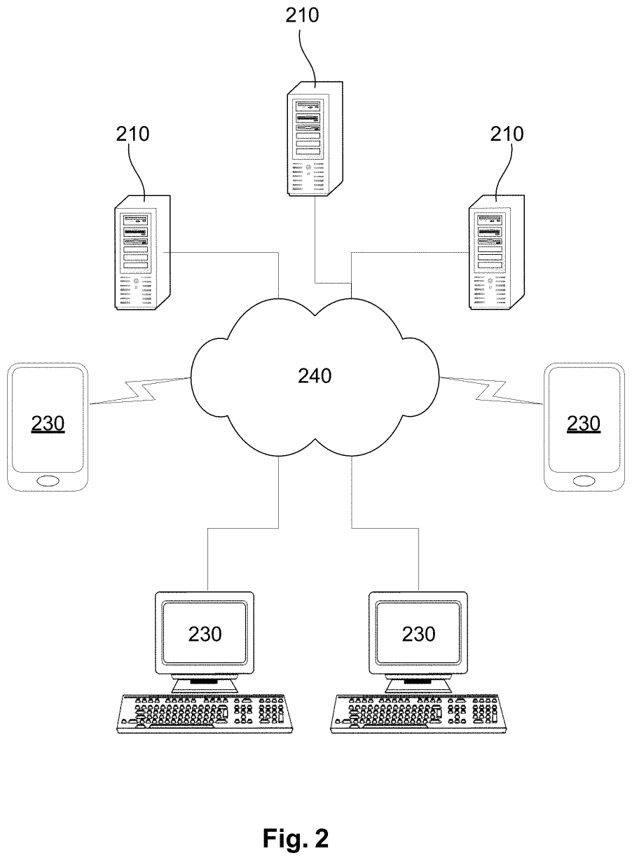

As mentioned above, in one example, the process is performed by one or more processing systems and client devices operating as part of a distributed architecture, an example of which will now be described with reference to FIG. 2.

In this example, a number of processing systems 210 are coupled via communications networks, such as the Internet 240, and/or one or more local area networks (LANs), to a number of client devices 230. It will be appreciated that the configuration of the networks 240 are for the purpose of example only, and in practice the processing systems 210 and client devices 230 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

In one example, each processing system 210 is able to analyze subject data and generate skill state indicators, providing these to a client device 230, allowing the client device 230 to display a visual representation, and/or generating recommendations that can then be displayed by the client device 230. The processing systems 210 can also analyze reference subject data and generate computational models. Whilst the processing system 210 is shown as a single entity, it will be appreciated that the processing system 210 can be distributed over a number of geographically separate locations, for example by using processing systems 210 and/or databases that are provided as part of a cloud based environment. However, the above described arrangement is not essential and other suitable configurations could be used.

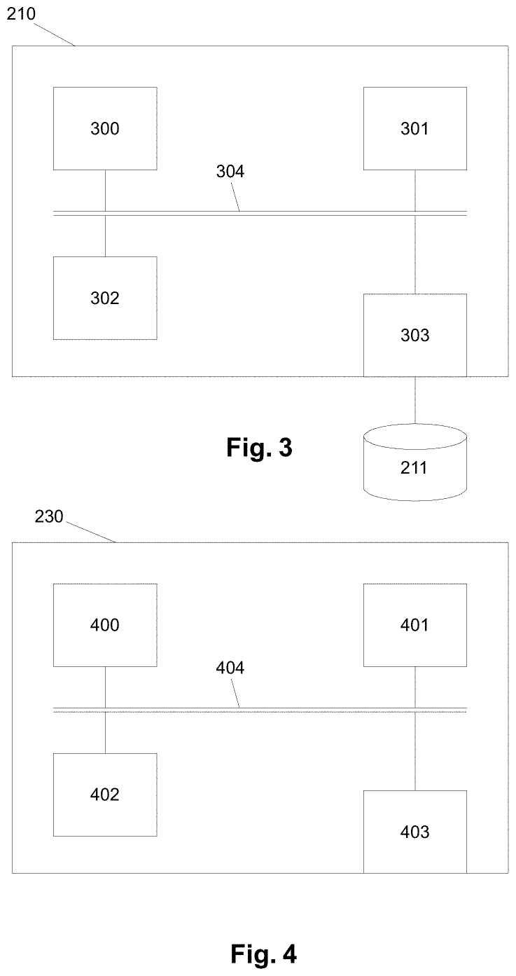

An example of a processing system 210 is shown in FIG. 3.

In this example, the processing system 210 includes at least one microprocessor 300, a memory 301, an optional input/output device 302, such as a keyboard and/or display, and an external interface 303, interconnected via a bus 304 as shown. In this example the external interface 303 can be utilized for connecting the processing system 210 to peripheral devices, such as the communications network 240, databases, other storage devices, or the like. Although a single external interface 303 is shown, this is for the purpose of example only, and in practice multiple interfaces using various methods (eg. Ethernet, serial, USB, wireless or the like) may be provided.

In use, the microprocessor 300 executes instructions in the form of applications software stored in the memory 301 to allow the required processes to be performed. The applications software may include one or more software modules, and may be executed in a suitable execution environment, such as an operating system environment, or the like.

Accordingly, it will be appreciated that the processing system 210 may be formed from any suitable processing system, such as a suitably programmed client device, PC, web server, network server, or the like. In one particular example, the processing system 210 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

An example of a client device 230 is shown in FIG. 4.

In one example, the client device 230 includes at least one microprocessor 400, a memory 401, an input/output device 402, such as a keyboard and/or display, and an external interface 403, interconnected via a bus 404 as shown. In this example the external interface 403 can be utilized for connecting the client device 230 to peripheral devices, such as the communications networks 240, databases, other storage devices, or the like. Although a single external interface 403 is shown, this is for the purpose of example only, and in practice multiple interfaces using various methods (eg. Ethernet, serial, USB, wireless or the like) may be provided.

In use, the microprocessor 400 executes instructions in the form of applications software stored in the memory 401 to allow communication with the processing system 210, for example, to allow subject data to be captured and uploaded to the processing systems 210 and to allow skill state indicators to be displayed to users of the client devices.

Accordingly, it will be appreciated that the client devices 230 may be formed from any suitable processing system, such as a suitably programmed PC, Internet terminal, lap-top, or hand-held PC, and in one preferred example is either a tablet, or smart phone, or the like. Thus, in one example, the client device 230 is a standard processing system such as an Intel Architecture based processing system, which executes software applications stored on non-volatile (e.g., hard disk) storage, although this is not essential. However, it will also be understood that the client devices 230 can be any electronic processing device such as a microprocessor, microchip processor, logic gate configuration, firmware optionally associated with implementing logic such as an FPGA (Field Programmable Gate Array), or any other electronic device, system or arrangement.

Examples of the processes for monitoring and improving social-emotional skills will now be described in further detail. For the purpose of these examples it is assumed that one or more processing systems 210 act to maintain subject data, generate and analyze metrics and generate skill state indicators, which can then be provided to the client devices 230, with resulting visualizations or recommendations being displayed by the client devices 230. In one example, to provide this in a platform agnostic manner, allowing this to be easily accessed using client devices 230 using different operating systems, and having different processing capabilities, input data and commands are received from the client devices 230 via a webpage, with resulting visualizations being rendered locally by a browser application, or other similar application executed by the client device 230.

The processing system 210 is therefore typically a server (and will hereinafter be referred to as a server) which communicates with the client device 230 via a communications network 240, or the like, depending on the particular network infrastructure available.

To achieve this the server 210 typically executes applications software for hosting webpages, as well as performing other required tasks including storing, searching and processing of data, with actions performed by the processing system 210 being performed by the processor 300 in accordance with instructions stored as applications software in the memory 301 and/or input commands received from a user via the I/O device 302, or commands received from the client device 230. It will also be assumed that the user interacts with the server 210 via a GUI (Graphical User Interface), or the like presented on the client device 230, and in one particular example via a browser application that displays webpages hosted by the server 210, or an App that displays data supplied by the server 210. Actions performed by the client device 230 are performed by the processor 400 in accordance with instructions stored as applications software in the memory 401 and/or input commands received from a user via the I/O device 402.

However, it will be appreciated that the above described configuration assumed for the purpose of the following examples is not essential, and numerous other configurations may be used. It will also be appreciated that the partitioning of functionality between the client devices 230, and the server 210 may vary, depending on the particular implementation.

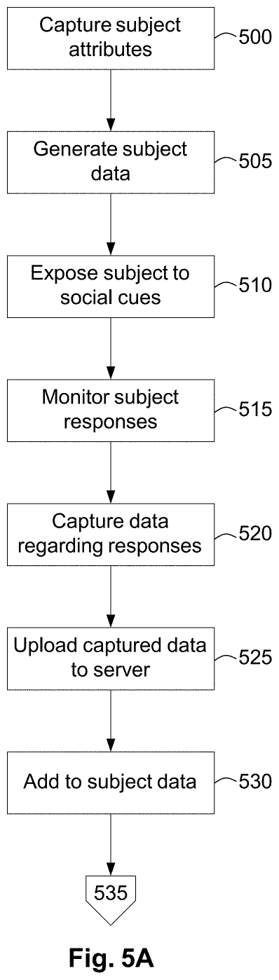

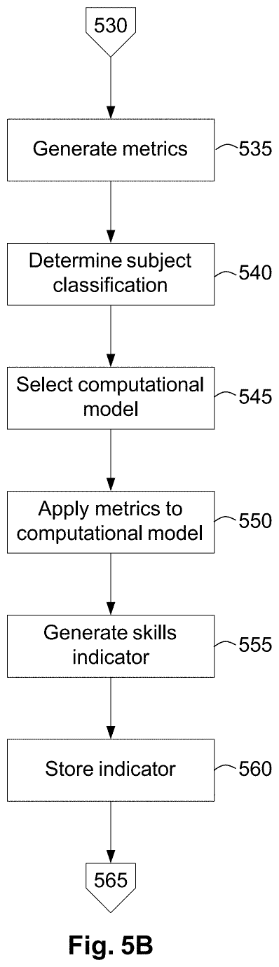

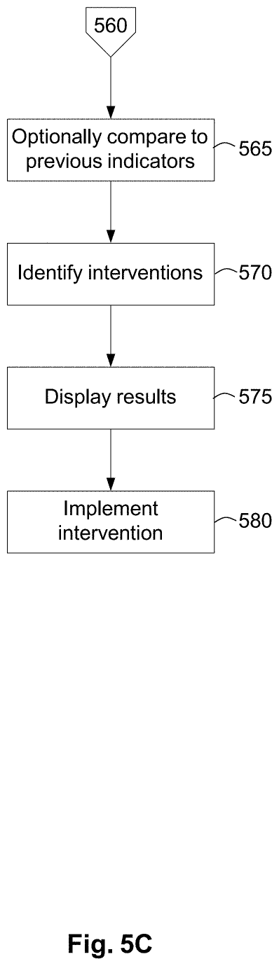

An example of the process for improving a subject's social-emotional skills will now be described in more detail with reference to FIGS. 5A to 5C.

In this example, at step 500 information regarding one or more subject attributes is determined. This is typically performed as a one-off process when a subject undergoes an initial assessment, but could also be repeated periodically, such as each time a measurement is performed, depending on the nature of the subject attributes. The subject attributes could be retrieved by the server 210 from a medical record, for example, by providing the server 210 with details of the subject, allowing this to be used to retrieve the attributes, or could be provided by having a user, such as the subject or a supervising practitioner, such as a teacher or clinician, enter the subject attributes via a suitable user interface presented on the client device 230. It will be appreciated that this could be achieved by presenting an application on the client device 230 and/or through a website hosted by the server 210.

At step 505, the server 210 generates subject data in the form of a particular record associated with the respective subject. As part of this process, a subject identifier is typically created and associated with the subject data. This could be a username, but more typically is a unique alphanumeric code, which can be used to anonymize the subject data as needed. The subject data may be stored in an encrypted database, with access permissions being defined for specific users, such as authorized clinicians, or the like, to thereby prevent unauthorized access to the subject data.

At step 510 the subject is exposed to one or more social cues, with subject responses being monitored at step 515, and data regarding the responses being captured at step 520. As previously mentioned, this might occur in a variety of ways depending on the preferred implementation. For example, the subject could be instructed to play a computer game using a client device 230, in which case the client device will monitor responses in accordance with user input commands provided by the subject as the game is played, thereby allowing the game play data to be captured.

Alternatively, the monitoring process could be observed manually, for example, by placing the subject in a scenario where the subject is exposed to social cues, such as in a classroom, home environment, or similar. In this case, responses are typically monitored by one or more individuals, who then provide feedback on the responses. This can be achieved by having the individual upload information via an interface displayed on the client device 230, for example by answering a series of set questions regarding the response to the social cues.

It will also be appreciated however that data could be captured in other appropriate manners. For example, sensors could be used to capture data, such as physiological data whilst the subject is exposed to the social cues. This would typically involve having the subject, or a supervising individual, attach the monitoring device to the subject in an appropriate manner, depending for example on the nature of the sensors. This could include simply having the subject wear the monitoring device, or could include attachment of electrodes to the subject. The sensor data is acquired, typically by filtering and digitizing signals received from the sensors, before being uploaded to the client device 230. As part of this process, the client device 230 can optionally process the received sensor data, for example to perform filtering and/or derive relevant parameters, such as a heartbeat, or the like. Such parameterization can reduce the volume of data that needs to be transferred to the server 210, although it will be appreciated that this is not essential, and alternatively the raw data could be transferred, depending on the preferred implementation.

At step 525, the client device 230 adds a subject identifier indicative of an identity of the subject to any captured data, allowing this to be uploaded to the server 210, and added to the subject data associated with the respective subject at step 530. Thus, the subject identifier is used to match captured data with pre-existing subject data stored in a database or elsewhere.

This process may require that the client device 230 authenticate the subject or individual controlling the data upload, for example by having them provide biometric information, such as a fingerprint, or respond to an authentication challenge, for example by providing a password, enter a PIN (personal identification number) or the like. The subject identifier can then be retrieved from local memory on the client device 230, retrieved from the monitoring device, or could be entered manually by a user. Additionally, the process may require the client device 230 display one or more questions to the subject, allowing the subject to respond and provide information regarding attributes, such as current symptoms, details of any food, beverage or medications consumed or the like. The client device may also interface with other sensing devices, such as weight scales, allowing other subject attributes to be captured as required.

It will be appreciated that this process could be performed for a subject undergoing assessment as well as reference subjects when the reference data is being collected to perform training of the computational model.

Once the subject data has been updated, at step 535 the subject data is analyzed allowing one or more metrics to be generated. The manner in which this is achieved will vary depending on the nature of the metrics and the subject data. For example, some of the metrics will simply be the subject data, and can therefore simply be retrieved from the subject data. For example, the metrics could include an indication of a score in the game, in which case this could be retrieved from the game play data forming part of the subject data. Alternatively, processing might be required, for example to derive a rating for a particular social-emotional skill, based on how well social cues were correctly identified by the subject. Similarly, heart rate variability may need to be calculated from heart rate data, or the like.

At step 540, the server 210 determines a subject classification. The classification is typically performed based on subject attributes, such as physical and/or demographic attributes. As these are typically relatively constant, the classification may only need to be performed a single time and retrieved from the subject data in subsequent iterations. Alternatively, this could be calculated each time, depending on the preferred implementation and/or nature of the classification performed. In general, the classifications are created by performing clustering of reference subjects, with this being performed to identify reference subjects having similar characteristics and/or who respond in a similar manner to therapeutic interventions. Once created, subjects can then be assigned to the classification by comparing relevant attributes to the attributes of individuals in the different clusters, and assigning the subject based on a closest match.

At step 545 the classification is used to select a computational model that is specific to the respective classification, with the calculated metrics then being applied to the computational model at step 550 to allow a skill state indicator to be calculated/generated at step 555 and stored at step 560. The manner in which this is performed will vary depending on the preferred implementation. For example, this might involve using relevant metrics, optionally together with one or more subject attributes, to form a feature vector, which is then applied to the computation model. The model analyses the feature vector, and calculates a skill state indicator, which is indicative of the social-emotional skill state of the subject. It will be appreciated that the technique(s) used will vary depending on the nature of the model, and as such techniques are known, these will not be described in further detail.

At this stage, the server 210 may also compare the current skill state indicator to one or more previous skill state indicators for the subject at step 565, allowing a change in skill state to be assessed. The skill state and any changes may also be used to identify potential interventions at step 570, although more typically the recommendations for the interventions are generated as part of the skill state indicator calculated using the computational model.

At step 575, the server 210 provides the skill state indicator to a client device 230, allowing the subject and supervisor to review results and proceed with implementing any recommended therapeutic interventions at step 580. The process can end at this point, although more typically the process is repeated, allowing a longitudinal trend of changes in skill state indicator to be used together with other accompanying information, such as details of interventions, to track progression of social-emotional skill states and the effectiveness of treatment. This in turn can feed back into the modelling, allowing further analysis to be used to increase the effectiveness of the computational models as more data is collected.

Although the above description has focused on the delivery of therapeutic interventions, it will be appreciated that this is not necessarily essential and in other examples, the techniques can be used in a purely diagnostic/monitoring framework. Thus, the system can be used for diagnosing, monitoring and/or treating conditions, such as autism, or other psychological or developmental conditions.

Accordingly, the above described system seeks to provide a machine learning based approach to guide therapeutic interventions and/or diagnosis or monitoring of the social-emotional skills of individuals. In one example, the system is configured to perform the analysis and provide recommendations for intervention that empowers children or other individuals to learn and apply social-emotional skills in an individually-tailored manner.

In one example, this can be achieved in conjunction with a SAS program, allowing interventions to be provided in a fun, self-directed and engaging manner. In one example, the above described system can help provide a scalable social technology allowing children worldwide to receive real-world coaching from a smartphone/smartwatch app that empowers self-identification and management of their emotions (Stage 1), enriches their social exchanges (Stage 2) with others in a privacy-protected manner, and alerts adults in real-time when children are experiencing emotional or physical stress e.g. bullying.

To help achieve these objectives, in one example the system can use a network of service-delivery organizations and research institutions to collect and analyze a wide range of subject data from a representative sample of children, parents and educators. Machine learning can then be used to derive computational models that can guide human behavior, and in particular offer interventions to improve mental health and safety.

In one example, this process can be implemented as part of the SAS program, allowing it to be optimized for the individual needs of different children to enhance intervention outcomes.

In this regard, whilst SAS empowers social-emotional resilience through physical program materials, computer game-play and adult-led monitoring/reward systems, a limitation is the availability, skill and capacity of adults to provide children with real-time feedback supporting skills practice. The above described systems seek to address this by using machine learning to more accurately assess and support children's social-emotional skill development through the use of AI.

In one example, the above described system can use wearable and mobile client devices with rich, layered, contextual subject data enabling children's independent social-emotional skills practice. Specifically, machine learning can be used to increase quality and quantity of skills practice through two stages, 1) coaching through physiological/activity/demographic data, and 2) multi-faceted real-time coaching and reporting including facial expression and conversational data.

The use of machine learning allows the system to provide targeted, personalized coaching (e.g. personalized assistance prompt on detection of high arousal). Machine learning can also allow for faster processing of pattern recognition within complex datasets for real-time analysis of skill development (analyzing rich multi-source data instead of relying on adult observational survey) and faster real-time feedback on user submitted content (e.g. facial emotion assessment on images). This also provides potential for less biased and more repeatable assessment of datasets.

In one example, the above described system can utilize different data streams to enhance the assessment performed, and use data across multiple sources to improve the discriminatory and advisory capabilities of the system. Examples of data that can be used include, but are not limited to, physiological data, such as heart rate and heart rate variability (via photoplethysmography (PPG)), Galvanic Skin Response (GSR), temperature; activity data, such as location, time, motion (via tri-axial accelerometer), and possible user-submitted classifications; and demographic data, including personal and health/sensitive information, such as age, gender, height, weight, racial background.

Additionally, the system can use conversational audio/video data, such as recordings of conversations between users and others; text, such as text entries and exchanges gathered as part of intervention, e.g. journaling activities or intervention-specific conversations; and facial imagery, such as self-generated and media-sourced images/videos of the subject and others.

Data can also be collected using a variety of different channels to support further levels of analysis of data including for example physiological responses and real-time social skills reactions and coaching, emergency response support, or the like.

Subject data can be acquired through computer game-play using an integrated reporting system that can collect game play data from users including elements of knowledge, accuracy, time, timing of use, choices of different pathways, monitoring and rewarding of skills usage in the ‘real world’, and personal reflections on skills practice activities.

Manually or physically, information can be collected on multi-source and multi-setting ratings of child skills (evaluation pre-post program), monitoring and rewarding skills usage in the ‘real world’, behavior and learning processes in group sessions, parent engagement and support needs, teacher engagement and support needs, intake information, such as diagnosis/diagnoses, developmental-, psychological-, medical- and/or family history, current behavior, medications, successful support strategies/academic achievement, or the like. In one example, as part of this, the system can collect data from research trials including physiological, observational and questionnaire data analyzed to evaluate program outcomes; in-built assessment tools used by SAS facilitators to capture individual child outcome data before, during and after SAS; impact of professional SAS practitioner training evaluated via pre-post training assessment measures; email and website user behavior analysis and user databases.

In one example, the machine learning approach could be configured to use physiological/activity/demographic data. This can be used to detect arousal levels across contexts (e.g. classroom vs. playground) cueing the system to offer user-support. Pattern identification in multi-source subject data can be used to personalize and improve cueing and support. Tracking multi-source subject data can be used to improve assessment accuracy, comprehensiveness, and provide real-time alerting (e.g. bullying alert), thereby improving individual outcomes, providing program evaluations and ultimately informing societal policies.

In another example, the machine learning approach could be configured to use conversational analysis, determining conversational quality and offering feedback. Data can be used to diarize speakers, assess pacing/pauses, volume/tonal variation, exchanges, or the like. This could include using delayed-response and real-time feedback possibilities.

In a further example, the machine learning approach could be configured to use emotional recognition. In this example, the system can detect facial expressions to enable the provision of feedback during learning and prompting during real-time coaching. This could include categorization of emotional expression through images/video data.

In a further example, the machine learning approach could be used to synthesize data streams and explore key relationships across complex data streams, synthesizing systems to produce multi-faceted real-time SAS coaching and reporting.

The subject data can include an analysis of a child's ability to correctly identify emotions, broken down by competence in identifying facial expression, voice-tone and body posture clues. The subject data could also include recommendations regarding emotions that should be focused on more in therapy, based on a child's performance in Level 1 of the SAS computer game, and can include data captured of a child's use/application of skills taught in the SAS computer game and therapeutic model as a whole, both at home and at school. This can be used to inform recommendations on which skills a child needs to practice more, or where parent-, teacher- or program facilitator expectations regarding frequency of skill usage may be too low or too high.

The system can perform auto-analysis of parent-, child-, SAS facilitator- and/or teacher responses to questionnaires, intake interview data captured and/or observational data captured to create a profile analysis of areas of social-emotional functioning strength and weakness for a child at the beginning of the program, at the end, and at 3- and 6-month follow-up.

The system can also determine areas where parents and teachers are in agreement regarding a child's competency in social-emotional functioning and where there is divergence/disagreement. This data informs initial and ongoing therapy planning for the child, together with recommendations for how parents, teachers and/or therapists can optimally support a child's social-emotional skill development.

In one example, through aggregate collection of questionnaire data across multiple children who access this program across the word—a parent, teacher, trained Secret Agent Society facilitator or insurance company could predict how successful Secret Agent Society will be for a family based on a child's age, diagnosis/diagnoses, IQ, medication use and other predictive variables (to be determined).

In one example, the above described arrangement can be used to provide guidance regarding therapeutic inventions, such as programs to improve the subject's social-emotional skills. This can include details of a treatment direction, such as areas of focus for the child and could inform both clinical decisions for therapeutic direction that have insurance/funding implications.

The system could be configured to provide diagnostic specific profiles, which can facilitate diagnosis, as well as service type specific profiles (e.g. private practice clients vs government health system clients vs school sector specific etc), which can assist service providers in configuring services to meet the needs of subjects.

Country-specific profiles could be used to generate intervention programs that match available services or other requirements for different countries, whilst individualized treatment pathways can be provided at a per child level. For example, this can be used to inform individualized software/UI/system for different children (automatic based on initial user behavior or user input of classification data), so that interaction with the computer game is tailored for each child's capabilities, including literacy levels. The system can be used to develop algorithms to identify early indicators of child safety concerns, allowing this to be based on information gathered directly from the child, rather than relying on adult reporting or observation, which can in turn aid in providing protection online or prioritization of services to support/intervene with family.

The system can be used to facilitate professional training, improving the effectiveness of training and translation of professional training to deliver SAS and evidence-based program implementation in general. The system can also be used to develop individualized digital learning experiences including selecting a suitable user interface, learning style, professional type, to best meet the needs of the individual participating in professional training.

The system can be used to ensure program fidelity and assist organizational implementation for professional training, clinical/educator program application, and child outcomes. This can provide information regarding program fidelity, individual clinician/educator performance, staff development needs, business efficiency ratings, individual child/family outcomes reporting to funding/regulators/marketing/insurers and community/school outcomes reporting to funding/regulators/marketing/insurers.

In one example, the system can be configured to generate facilitator report data, which can be used by a therapist or teacher. Such a report can aggregate data on:
children's computer game completion and performance with recommendations provided on target emotions/skills that therapists or teachers should spend longer on with a group of children (small group or class)
the quality of children's completion of social-emotional skills practice/application tasks at home and at school (missions), so facilitator can follow-up with a child/children (and/or their parents) who is/are not practicing the social-emotional skills introduced in the computer game in real life (or those who are practicing the skills, but are struggling with them) and offer them additional help with this if needed.
the frequency with which child group members/class members is/are practicing skills introduced through the Secret Agent Society Computer Game and the therapeutic model as a whole.
the effectiveness of Secret Agent Society in improving children's emotion regulation skills, behavior and social interaction skills, and how well these improvements are maintained 3- and 6-months after the program ends. This data is to include interview, observational assessment data and questionnaire data from parents, teachers, facilitators and/or children.

The system can also be configured to provide an organizational report including:
reporting on number of children who have gone through the program
the program's effectiveness for the cohort of children who have gone through it within a certain time frame (see facilitator report data section above)
demographics associated with children/families who are good versus poor treatment responders, to inform treatment planning/who the program would be best suited for in the future at their agency/organization
the fidelity with which trained program facilitators delivered the program (number of program activities completed, relative to the number of activities intended per session) and
the specific activities delivered by trained facilitators with low or high treatment fidelity/program adherence. This can guide what future supervision sessions around program delivery should focus on, and the support a group of trained facilitators may wish to get from the Social Skills Training Institute to improve program fidelity and effectiveness.

In one embodiment, the method and apparatus for improving social-emotional skills in a biological subject includes one or more computers having one or more processors and memory (e.g., one or more nonvolatile storage devices). In some embodiments, memory or computer readable storage medium of memory stores programs, modules and data structures, or a subset thereof for a processor to control and run the various systems and methods disclosed herein. In one embodiment, a non-transitory computer readable storage medium having stored thereon computer-executable instructions which, when executed by a processor, perform one or more of the methods disclosed herein.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

What is claimed is:

1. A method for improving social-emotional skills in a biological subject, the method comprising:
acquiring subject data, the subject data being at least partially indicative of a subject response to one or more social cues;
deriving one or more metrics from the subject data, the one or more metrics including at least one response metric at least partially indicative of the subject response;
in one or more electronic processing devices, applying the one or more metrics to at least one computational model to generate a skill state indicator relating to a social-emotional skills state of the subject, the at least one computational model embodying a relationship between social-emotional skill states and the one or more metrics, wherein the at least one computational model is obtained by applying machine learning to reference metrics derived from reference subject data measured for one or more reference subjects; and,
using the skill state indicator to perform a therapeutic intervention to thereby improve the social-emotional skills of the subject.

2. The method of claim 1 further comprising:
generating subject data at least in part based on an assessment of an ability of the subject to recognize the social cues.

3. The method of claim 1 further comprising:
exposing the subject to one or more social cues;
monitoring subject responses; and,
using the subject responses to at least one of:
assess the ability of the subject to recognize the social cues; and,
train the subject to recognize the social cues.

4. The method of claim 1 further comprising:
having the user interact with a computer game implemented using a suitably programmed computer system, the computer game being configured to teach social-emotional skills to the subject;
using the computer game to generate game play data; and,
using the game play data to derive one or more response metrics.

5. The method of claim 4 further comprising:
in a computer implementing the computer game;
presenting the subject with one or more social cues;
ascertaining a subject responses to the social cues in accordance with user input commands;
generating game play data indicative of at least one of:
the user input commands;
the subject responses;
an accuracy of the subject responses;
a speed of the subject responses;
a degree of participation;
demonstrated elements of knowledge; and,
selected choices; and
at least one of:
progressing the game based on the subject response; and,
selectively displaying feedback to the subject based on the subject response.

6. The method according to claim 1 further comprising:
using one or more sensors to measure subject attributes, optionally while the subject is interacting with a computer game implemented using a suitably programmed computer system, the computer game being configured to teach social-emotional skills to the subject playing the computer game; and,
generating one or more metrics using measured subject attributes.

7. The method of claim 1, wherein the subject data includes at least one of:
game play data collected while the subject is interacting with a computer game implemented using a suitably programmed computer system, the computer game being configured to teach social-emotional skills to the subject playing the computer game;
details of therapeutic interventions performed on the subject;
results of therapeutic interventions performed on the subject;
results of one or more assessments of social-emotional skills of the subject;
ratings of one or more social-emotional skills of the subject;
responses to one or more social cues;
subject attributes including one or more of:
diagnosed psychological and/or developmental disorders;
demographic attributes;
physiological attributes;
physical attributes;
psychological attributes;
nutritional information relating to nutrition of the subject; and,
medication information relating to medication administered to the subject;
recordings of at least one of:
conversations;
social interactions; and,
responses to social cues; and,
journal records.

8. The method of claim 1 further comprising:
acquiring subject data by at least one of:
having the subject interact with a computer game implemented using a suitably programmed computer system, the computer game being configured to teach social-emotional skills to the subject playing the computer game;
querying a subject medical history;
receiving sensor data from a sensing device;
performing an assessment of responses to social cues;
using questionnaires or forms completed by the subject;
using questionnaires or forms completed by one or more entities delivering therapeutic interventions; and,
using questionnaires or forms completed by one or more entities assessing social-economic skills of the subject.

9. The method of claim 1, wherein the one or more metrics include at least one of:
at least one subject attribute metric indicative of one or more subject attributes of the subject;
at least one physiological metric indicative of one or more physiological attributes of the subject;
at least one psychological metric indicative of one or more psychological attributes of the subject;
at least one physical characteristic metric indicative of one or more physical attributes of the subject;
at least one demographic metric indicative of one or more demographic attributes of the subject;
a physical activity metric indicative of physical activity of the subject; and,
at least one nutritional metric indicative of nutrition ingested by the subject;
at least one medication metric indicative of medication administered to the subject;
at least one pre-therapy metric; and,
at least one post-therapy metric.

10. The method of claim 9, wherein at least one of the physiological attributes includes at least one of:
heart rate;
heart rate variability;
galvanic skin response;
breathing;
temperature;
a blood pressure;
facial expression;
gaze;
speech; and
a presence, absence or degree of one or more disorder states,
wherein the physical attributes includes at least one of:
a subject age;
a subject height;
a subject weight;
a subject sex; and
a subject ethnicity,
wherein the psychological attributes includes at least one of:
happy;
sad;
anxious;
angry;
tired; and,
a mental state including at least one of:
engaged;
focused;
disinterested; and
bored, and
wherein the physical activity attributes include at least one of:
activity type;
activity intensity;
activity duration;
activity location;
activity time; and
amount of movement.

11. The method of claim 1, wherein at least one of:
the social-emotional skills state includes at least one of:
a diagnosis of a presence, absence or degree of a condition that impacts on social-emotional skills of a subject;
a competency level for one or more social-emotional skills;
a change in competency level for one or more social-emotional skills;
a comparison between a competency level and an expected competency level for one or more social-emotional skills; and
a ranking of social-emotional skills; and
the skill state indicator includes an indication of at least one of:
a score for one or more social-emotional skills, the score being indicative of a competency level;
results of a comparison between a competency level and an expected competency level for one or more social-emotional skills;
a change in competency level for one or more social-emotional skills;
a list of one or more social-emotional skills that require improvement;
an ordered list indicative of a ranking of social-emotional skills;
a recommendation for improving one or more social-emotional skills;
one or more tasks designed to improve one or more social-emotional skills;
an intervention program for improving one or more social-emotional skills;
medication for improving one or more social-emotional skills; and,
a diagnosis of a presence, absence or degree of a condition that impacts on social-emotional skills of a subject.

12. The method of claim 1, wherein the therapeutic intervention includes at least one of:
controlling a computer game implemented using a suitably programmed computer system, the computer game being configured to teach social-emotional skills to the subject playing the computer game;
training the subject;
training the subject in accordance with a recommendation;
having the subject interact with a computer game implemented using a suitably programmed computer system, the computer game being configured to teach social-emotional skills to the subject playing the computer game;
having the subject perform one or more intervention tasks;
having the subject complete a treatment program; and,
administering medication.

13. The method of claim 1, wherein the method includes generating a report indicative of at least one of:
a usage of one or more therapeutic interventions;
an adherence to one or more therapeutic interventions;
an effectiveness of one or more therapeutic interventions;
an effectiveness of one or more entities delivering therapeutic interventions;
an effectiveness of one or more entities assessing social-emotional skills;
an effectiveness of therapeutic interventions for different classifications of subject; and,
a comparison of the effectiveness of different therapeutic interventions.

14. The method of claim 1 further comprising:
assigning the subject to a classification, optionally at least in part using subject attributes; and,
applying the one or more metrics to at least one computational model associated with the classification, the at least one computational model embodying a relationship between social-emotional skills and the one or more metrics for reference subjects assigned to the classification.

15. The method of claim 1 further comprising:
comparing at least one current metric determined for the subject and at least one previous metric determined for the subject; and,
using results of the comparison to track at least one of:
change in social-emotional skills of the subject; and,
effectiveness of therapeutic interventions.

16. The method of claim 1, wherein the method includes:
for each of a plurality of reference subjects:
acquiring reference subject data at least partially indicative of:
a reference subject response to one or more social cues; and,
an assessment of a reference social-emotional skills state for the reference subject; and,
deriving one or more reference metrics from the reference subject data, the one or more reference metrics including at least one response reference metric at least partially indicative of the reference subject response; and,
in one or more electronic processing devices, using the reference metrics and the assessed reference social-emotional skills state to train at least one computational model so that the at least one computational model embodies relationships between different social-emotional skill states and the one or more metrics.

17. A method for calculating at least one computational model, the at least one computational model being used for generating a skill state indicator relating to a social-emotional skills state of a biological subject, the method comprising:
in one or more electronic processing devices:
for each of a plurality of reference subjects:
acquiring reference subject data at least partially indicative of:
a reference subject response to one or more social cues; and,
an assessment of a reference social-emotional skills state for the reference subject; and,
deriving one or more reference metrics from the reference subject data, the one or more reference metrics including at least one response reference metric at least partially indicative of the reference subject response; and,
using the reference metrics and the assessed reference social-emotional skills states to train the at least one computational model so that the at least one computational model embodies relationships between different social-emotional skill states and the one or more metrics.

18. The method of claim 17, wherein the one or more processing devices is configured to:
select a plurality of reference metrics;
train at least one computational model using the plurality of reference metrics;
test the at least one computational model to determine a discriminatory performance of the model; and,
if the discriminatory performance of the model falls below a threshold, at least one of:
selectively retrain the at least one computational model using a different plurality of reference metrics; and,
train a different computational model.

19. The method of claim 17 further comprising:
selecting a plurality of combinations of reference metrics;
training a plurality of computational models using each of the combinations;
testing each computational model to determine a discriminatory performance of the model; and,
selecting the at least one computational model with the highest discriminatory performance for use in determining a mental state indicator indicative of a mental state.

20. The method of claim 17 further comprising:
determining one or more reference subject attributes from the reference subject data;
training the at least one computational model using the one or more reference subject attributes; and
assigning the reference subjects to classifications and training the at least one computational model using the classifications, so that the at least one computational model embodies a relationship between social-emotional skills and the one or more metrics for reference subjects assigned to a respective classification.
